(19) United States
(12) Patent Application Publication
(10) Pub. No.: US 2020/0384252 A1
(43) Pub. Date: Dec. 10, 2020

(54) SYSTEM AND METHOD FOR APPLYING TREATMENT TO A TREATMENT ZONE

(71) Applicant: HighQ Services, LLC, Cedar Park, TX (US)

(72) Inventor: BABAK R SAMANI, Cedar Park, TX (US)

(21) Appl. No.: 16/896,385

(22) Filed: Jun. 9, 2020

Related U.S. Application Data
(60) Provisional application No. 62/859,199, filed on Jun. 10, 2019.

(51) Int. Cl. A61M 35/00; A61K 9/00; A61K 9/70

(52) U.S. Cl. CPC A61M 35/003; A61K 45/06; A61K 9/7007; A61K 9/0012

(57) ABSTRACT

A treatment kit includes a plurality of treatment applicators having a shape indicative of a nail. A first treatment applicator of the plurality of treatment applicators has a first size. A second treatment applicator of the plurality of treatment applicators has a second size. The first size is different from the second size. Each treatment applicator of the plurality of treatment applicators includes a substrate, a treatment layer coupled to the substrate, and a backing coupled to the treatment layer. The treatment layer includes a treatment agent.

### CROSS-REFERENCE TO RELATED APPLICATION(S)

This application claims benefit of U.S. Provisional Application No. 62/859,199, filed Jun. 10, 2019, which is incorporated herein by reference in its entirety.

### FIELD OF THE DISCLOSURE

This disclosure, in general, relates to systems and methods for applying treatment agents to a treatment zone.

### BACKGROUND

Nails, such as fingernails and toenails, are an important part of human anatomy. While providing some protection to the digits, nails also have an aesthetic aspect. Further, the appearance of nails can be indicative of various health conditions, such as vitamin deficiencies or infection.

In particular, nails can be infected with funguses and other infectious agents. Further nails can be dry or brittle as a result of nutritional deficiencies or other disease conditions. In such case, the nails lose their desirable aesthetic and can break easily, causing pain and other discomforts.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of a foot and an example set of nail treatment applicators.

FIG. 2 and FIG. 3 include cross-sectional illustrations of example nail treatment applicators.

FIG. 4 includes an illustration of an example packet including a set of mail treatment applicators.

FIG. 5 includes an illustration of a box of packets of nail treatment applicators.

FIG. 6, FIG. 7, and FIG. 8 include illustrations of example shapes of treatment applicators.

FIG. 9 includes a block flow diagram of an example method for treating the treatment zone.

FIG. 10 includes an illustration of an example treatment applicator.

FIG. 11 includes an illustration of a toe to which an example treatment applicator is applied.

FIG. 12, FIG. 13, FIG. 14, FIG. 15, FIG. 16, and FIG. 17 include an illustrations of an example treatment applicators.

The use of the same reference symbols in different drawings indicates similar or identical items.

### DETAILED DESCRIPTION

In an embodiment, a treatment applicator includes a substrate and a treatment layer adhered to the substrate. The treatment layer includes a treatment agent, such as an antifungal agent. In particular, the treatment applicators can be sized to fit a toenail and can have a similar size and shape to a toenail. A plurality of treatment applicators can be included in a packet. Each packet can include treatment applicators having different sizes. In a particular example, each of the treatment applicators of the plurality are secured to a card or strip and can be peeled or removed from the strip to be applied to a nail, such as a toenail. Alternatively, each of the treatment applicators can be secured to a separate backing. The plurality of packets of treatment applicators can be included in a box.

In an example, a user can open a box of treatment applicators and remove a packet that includes a plurality of treatment applicators of different sizes. The user can select a size corresponding to the size of a toenail, peel the selected treatment applicator, and apply it to the similarly sized toenail. The process can be repeated for other toenails, selecting an appropriately sized treatment applicator and applying the selected treatment applicator to the similarly sized toenail.

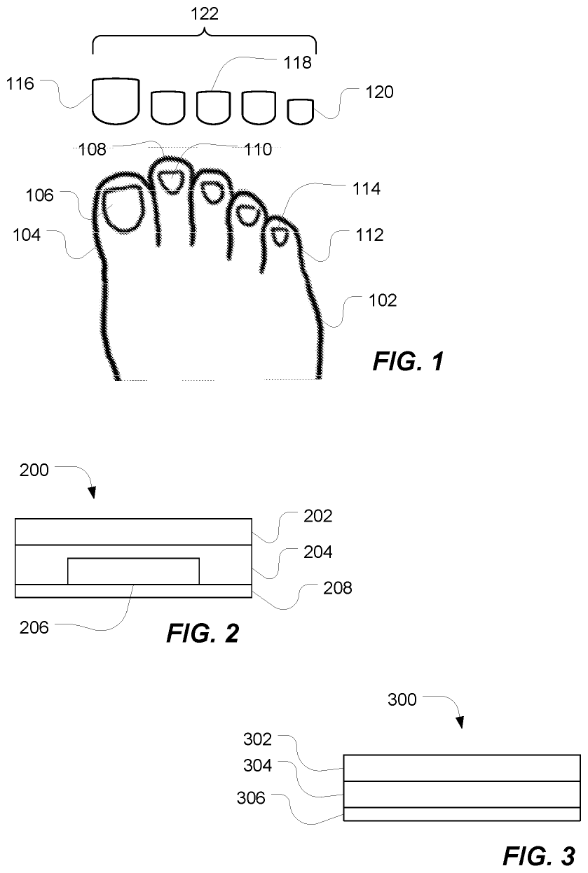

FIG. 1 includes an illustration of example treatment applicators. A foot 102 includes a big toe 104 that generally has a larger toenail 106 than other toes. Middle toes 108 are similarly sized and have similarly sized toenails 110. Often, a little toe 112 includes a smaller toenail 114. A set 122 of treatment applicators can be provided to treat toenails 106, 110 and 114. For example, the set 122 of treatment toenails can include two, three, four, or five treatment applicators. In another example, the set 122 of treatment applicators can include 10 treatment applicators. As illustrated, the set 122 of treatment applicators includes five treatment applicators of different sizes.

In an example, the set 122 of treatment applicators includes a larger treatment applicator 116 and a small treatment applicator 120. Further, the set 122 of treatment applicators can include a middle-sized treatment applicator 118. As such, the set 122 includes treatment applicators of at least two different sizes, such as at least three different sizes. Generally, the set 122 includes not greater than five different sizes of treatment applicators.

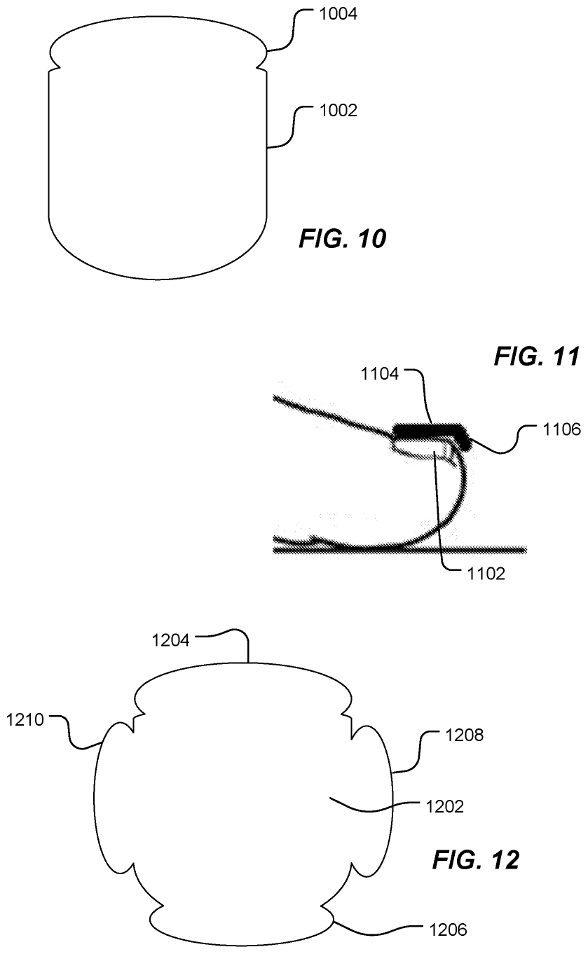

The treatment applicator can have the illustrated shape. In another example, the treatment applicator can have a shape, for example, as illustrated in FIG. 10. For example, the treatment application 1002 can further include a flap 1004. As illustrated in FIG. 11, the treatment applicator 1104 can be applied over a toenail 1102 and a flap 1106 can be folded over the front of the toenail. The flap can be characterized by indent cuts between the flap and main body, side slits, perforations, scoring, or pre-formed folds, or any combination thereof.

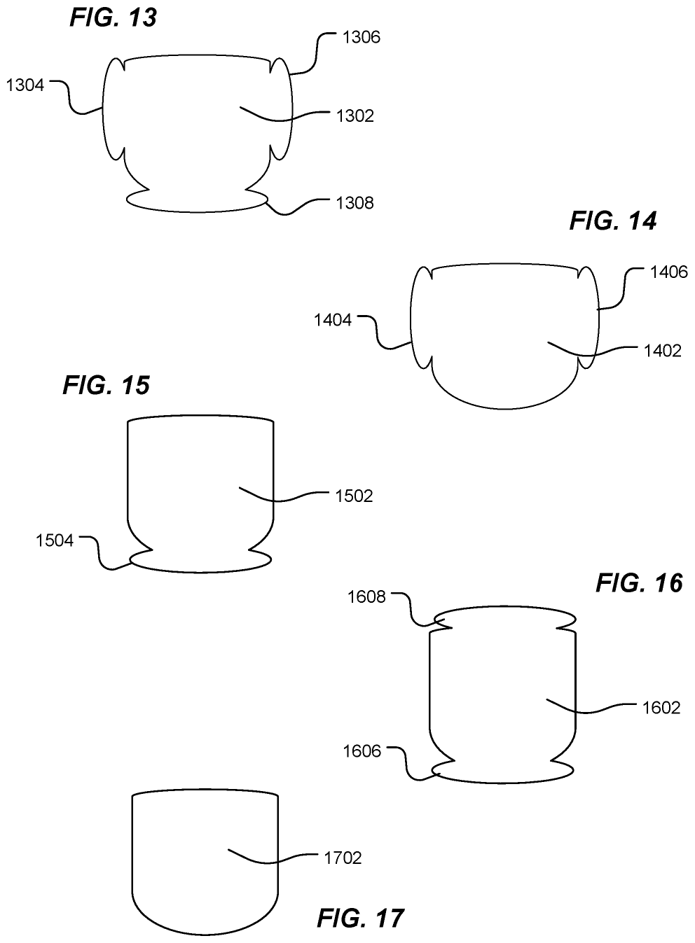

In another example, the treatment applicator can have side flaps and rear flaps. For example, as illustrated in FIG. 12, a treatment applicator 1202 can have a front flap 1204 to fold over a front of the toenail. The treatment applicator 1202 can further include a rear flap 1206 to secure the treatment applicator 1202 to a bed of the toenail. In addition, the treatment applicator 1202 can include side flaps 1208 or 1210 to secure the treatment applicator over the skin at the edges of the toenail.

In alternative examples, a treatment applicator 1302 can have side flaps 1304 and 1306 and a rear flap absent a front flap, as illustrated at FIG. 13. A treatment applicator 1402 can have side flaps 1404 and 1406 absent a front flap or a rear flap, as illustrated at FIG. 14. A treatment applicator 1502 can have only a rear flap 1504, as illustrated at FIG. 15. A treatment applicator 1602 can have a front flap 1608 and a rear flap 1606, as illustrated at FIG. 16. Alternatively, a treatment applicator 1702 may be free of flaps, as illustrated at FIG. 17.

Each of the treatment applicators can include a substrate to which a treatment layer is adhered. The treatment layer can include a treatment agent to treat a treatment zone, such as a toenail. In an example, the treatment layer includes a treatment agent, such as an antifungal agent, a transfer agent, or a health promoting agent.

In an example, the antifungal agent includes lema oil, alkyl dimethyl benzyl ammonium chloride, onymyrrhe, myrrh extract, byungpul extract, manuka oil, tea tree oil, zinc undecylenate or undecylenic acid, mentholated ointments such as menthol, eucalyptus oil, and camphor, snakeroot extract, lavender oil, petitgrain oil, clary sage oil, ylang ylang oil, jasmine oil, oregano oil, garlic extracts, or any combination thereof. For example, the antifungal agent can include lema oil, alkyl dimethyl benyl ammonium chloride, onymyrrhe, myrrh extract, byungpul extract, manuka oil, tea tree oil, or any combination thereof.

The transfer agent can alter the treatment zone, such as a toenail, to permit faster transfer of another treatment agent, such as the antifungal agent, through a toenail, toenail bed, or surrounding skin. In an example, the transfer agent includes propylene glycol, glycerin, mineral oil, a surfactant such as sodium dodecylbenzene sulfonate, or any combination thereof.

A health promoting agent can further assist in improving the health of the toenail or the nail bed by providing vitamins or other promoting agents. For example, the health promoting agent can include urea, salicylic acid, vitamin E, jojoba oil, coconut oil, olive oil, sweet almond oil, rice bran oil, shea butter, avocado oil, bee wax, lemon oil, argan oil, lanolin, aloe vera, or any combination thereof.

The treatment agent is included in the treatment layer to contact or directly contact the treatment zone, such as the toenail. The treatment layer can be an absorbent layer, such as a gauze or foam layer, that is secured to the substrate and secured in proximity to or adjacent to the toenail. Alternatively, the treatment agent can be included in a treatment layer that further has tacky or sticky properties to adhere to the toenail.

For example, FIG. 2 includes an illustration of an example treatment applicator 200 that includes a substrate layer 202. An adhesive layer 204 is secured to the substrate layer 202, and an absorbent material 206 is adhered to the adhesive layer 204. The treatment layer 206 can be sized smaller than adhesive layer 204 when viewed in cross-section to leave portions of the adhesive layer 204 exposed, allowing those portions to adhere to a surface, such as a toenail or skin on the edge of the toenail. In an example, the adhesive layer 204 can be included on flaps. A removable backing 208 is secured to the adhesive layer 204 and protects the adhesive layer 204 and the treatment layer 206 from exposure. The treatment agent can be disposed in the treatment layer 206.

The substrate 202 can be formed of a polymeric film. For example, substrate 202 can include polyethylene, polypropylene, poly butadiene, an acrylonitrile polymer, polystyrene, a block copolymer such as acrylonitrile butadiene styrene (ABS), ethylene-propylene monomer (EPM), ethylene propylene butadiene copolymer (EPDM), polyisoprene, polyacrylate, polyamide, or any combination thereof. The substrate 202 can further include fillers such as particulate filters or fibrous fillers. Optionally, such fillers can enhance strength.

In an example, the adhesive layer 204 includes a pressure sensitive adhesive. An example pressure sensitive can include an acrylic, butyl rubber, ethylene-vinyl acetate, natural rubber, nitriles, silicone rubbers, styrene block polymers, styrene-ethylene/butylene-styrene (SEBS), styrene-ethylene/propylene (SEP), styrene-isoprene-styrene (SIS), vinyl ethers, or any combination thereof. The adhesive layer 204 can further include a tackifier. An example tackifier includes rosin derivates, terpenes and modified terpenes, aliphatic, cycloaliphatic or aromatic resins (C5 aliphatic resins, C9 aromatic resins, or C5/C9 aliphatic/aromatic resins), hydrogenated hydrocarbon resins, terpene-phenol resins (TPR), or any combination thereof.

In an example, the treatment layer 206 is a sponge material or gauze material. For example, the treatment layer 206 can be formed of a fibrous mat using natural or polymer fibers. In another example, the treatment layer can include a sponge material, such as a natural or artificial sponge.

The backing 208 can include a wax paper or a polymer film. In an example, a polymer film can include a silicone, fluoropolymer, or other nonstick polymeric material. In particular, the backing 208 is easily separated from the adhesive layer 204.

In an example process, a substrate 202 can be coated with an adhesive 204. A treatment layer 206 having a width smaller than the adhesive layer 204 or planar dimension smaller than the adhesive layer 204 can be applied over the adhesive layer 204. A backing 208 can be applied to the treatment layer 206 and exposed adhesive layer 204.

In another embodiment, the treatment agent is incorporated into a tacky or sticky layer that adheres to the treatment zone, such as the toenail. For example, as illustrated in FIG. 3, a treatment applicator 300 includes a substrate 302, the treatment layer 304, and a backing 306.

The substrate 302 can be formed of a polymeric film. For example, substrate 202 can include polyethylene, polypropylene, poly butadiene, an acrylonitrile polymer, polystyrene, a block copolymer such as acrylonitrile butadiene styrene (ABS), ethylene-propylene monomer (EPM), ethylene propylene butadiene copolymer (EPDM), polyisoprene, polyacrylate, polyamide, or any combination thereof. The substrate 202 can further include fillers such as particulate filters or fibrous fillers. Optionally, such fillers can enhance strength.

The treatment layer 304 can include a tacky material such as a rubber and optionally, a tackifier and can incorporates the treatment agents. In an example, the tack material can include an acrylic, butyl rubber, ethylene-vinyl acetate, natural rubber, nitriles, silicone rubbers, styrene block polymers, styrene-ethylene/butylene-styrene (SEBS), styrene-ethylene/propylene (SEP), styrene-isoprene-styrene (SIS), vinyl ethers, or any combination thereof. The adhesive layer 204 can further include a tackifier. An example tackifier includes rosins derivates, terpenes and modified terpenes, aliphatic, cycloaliphatic or aromatic resins (C5 aliphatic resins, C9 aromatic resins, or C5/C9 aliphatic/aromatic resins), hydrogenated hydrocarbon resins, terpene-phenol resins (TPR) or any combination thereof. Treatment agents such as those described above can be incorporated into the tacky material.

The backing 306 is configured to be removable from the treatment layer 304 while leaving the treatment layer 304 secured to the substrate 302. The backing 306 can include a wax paper or a polymer film. In an example, a polymer film can include a silicone, fluoropolymer, or other nonstick polymeric material.

In an example, a treatment layer 304 including a tacky substance can be applied over the substrate 302. The backing 306 can be applied over the treatment layer.

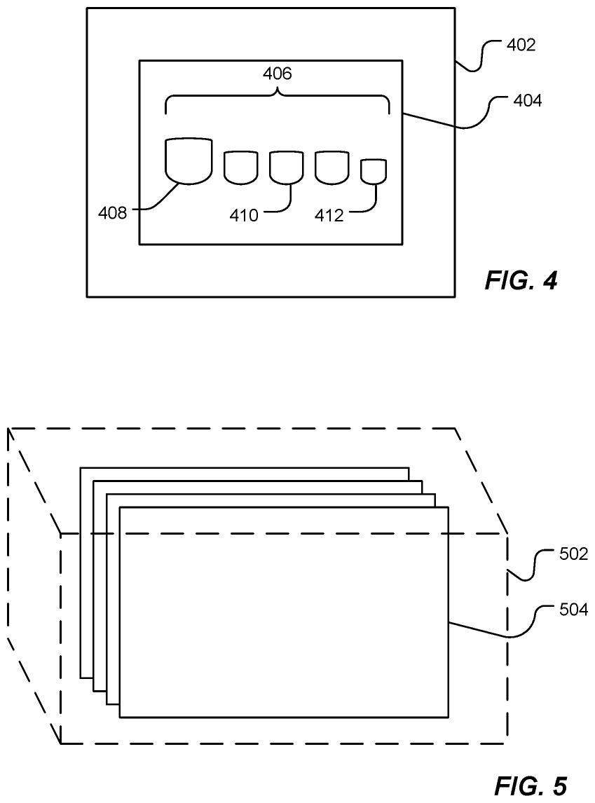

A plurality of treatment applicators can be included in a packet. For example, as illustrated in FIG. 4, a packet 402 can include a set of treatment applicators 406. Each of the treatment applicators is sized and shaped to fit a toenail. In a particular example, the set 406 of treatment applicators can be included on a card or strip 404. For example, the card or strip 404 can be the backing of the treatment applicators of the set 406. When disposed on the card, the treatment applicators can be arranged for example from largest to smallest. Alternatively, each treatment applicator of the set 406 can be separate and each can include its own backing.

The set 406 of treatment applicators includes a plurality of treatment applicators of at least two different sizes. In an example, the set 406 of treatment applicators includes treatment applicators of three different sizes. As illustrated, the set 406 of treatment applicators includes five treatment applicators including one large treatment applicator 408, three medium treatment applicators 410, and a smaller treatment applicator 412.

The packets of treatment applicators can be supplied in a box. As illustrated in FIG. 5, a box 502 includes a plurality of treatment applicator packets 504. In particular example, the boxes of treatment applicators can be provided that have treatment applicators of different ranges of size. For example, a box can be prepared that has treatment applicators sized to treat large feet. In such an example, the treatment applicators of different sizes within the packets are in a range of sizes that fit toenails of larger feet. In another example, a box can include a range of sizes of treatment applicators that fit smaller feet. In particular, boxes can be prepared that have ranges of sizes in small, medium, or large ranges.

In another embodiment, treatment applicators can be prepared that have shapes and sizes corresponding to various recognizable outlines. For example, the treatment applicators can be prepared in the shape of an animal or the shape of the tool. In particular, a treatment applicator can be formed in the shape of an animal. For example, treatment applicators can be included that are shaped in the form of easily recognized animals. For example, a set of treatment applicators could be in the shape of a mouse, cat, and dog. In another example, treatment applicators could be in the shape of wild animals, such as turtles, elephants, giraffes, bears, tigers, lions, or various other animals. In an additional example, a set of treatment applicators can be the shape of a variety of farm animals, such as chickens, pigs, cows, horses, or other farm animals.

Each of the differently shaped treatment applicators can be configured to treat a different type of wound. For example, the treatment applicator of a smaller animal could be configured to treat small puncture wounds, the treatment of a medium-size animal could be configured to treat a cut, whereas a larger animal-shaped treatment applicator could be used for treating scrapes.

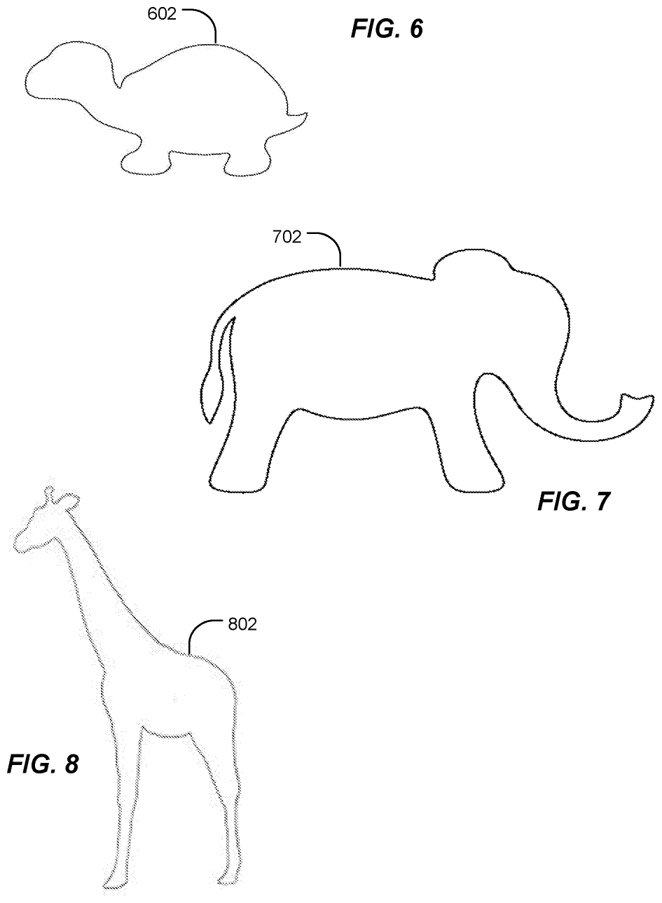

Each of the differently shaped treatment applicators can also include different treatment agents. As such, the treatment agent incorporated into each of the differently shaped treatment applicators can be different depending upon its purpose. For example, as illustrated in FIG. 6, the treatment applicator can have a turtle shape. As illustrated in FIG. 7 a treatment applicator can have an elephant shape, and as illustrated in FIG. 8, a treatment applicator can have a giraffe shape.

In an example, the turtle-shaped treatment applicator can be used to treat small puncture wounds and can optionally, include a treatment agent, such as an antibiotic agent to prevent infection in the puncture wound. Similarly, a treatment applicator shaped is a giraffe can be used to treat cuts and have antibacterial agents and optionally salves to enhance the healing of a cut. In contrast, the elephant-shaped treatment applicator could be used to treat scrapes that minimally penetrate the skin. In an example, the elephant-shaped applicator can include salves to maintain an environment around the skin that encourages quick healing. While the example uses a turtle, elephant, and giraffe, other animal shapes, tools, or recognizable items could indicate a purpose of the treatment applicator. While illustrated as a turtle, elephant, and giraffe, other differently shaped treatment applicators can take the form of other easily recognizable shapes.

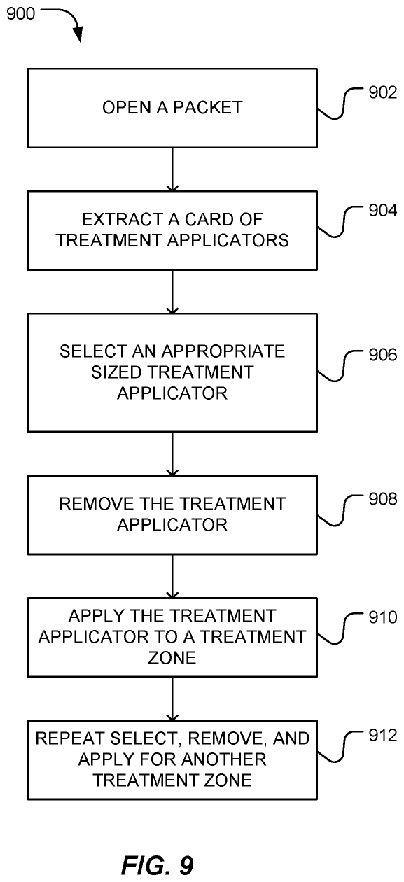

FIG. 9 includes an illustration of an example method 900 for treating a treatment zone. As illustrated at 902, a packet is opened. The packet can include a plurality of treatment applicators. For example, the treatment applicators can be adhered to a treatment card acting as a backing. The plurality of treatment applicators can include treatment applicators of different size.

The card can be extracted from the open packet, as illustrated at 904. Alternatively, each treatment applicator can be separate from the other treatment applicators and can include its own individual backing. When disposed on the card, the treatment applicators can be arranged for example from largest to smallest.

As illustrated at 906, an appropriately sized treatment applicator can be selected. For example, when the treatment applicators are configured to treat a treatment zone, such as a toenail, a treatment applicator can be selected that fits an associated toenail. For example, the larger treatment applicator can be selected to treat a big toe, whereas a smaller treatment applicator can be selected to treat the pinky toe or small toe.

As illustrated at 908, the selected treatment applicator can be peeled or removed from the backing, such as the card, and can be applied to the treatment zone, such as a toenail, as illustrated at 910. For example, when located on the card, the selected treatment applicator can be peeled from the card and applied to toenail.

As illustrated at 912, the process can be repeated to treat other treatment zones, such as other toenails. For example, the select, remove, and apply steps can be repeated for each toe of the foot.

In a first aspect, a treatment kit includes a plurality of treatment applicators having a shape indicative of a nail. A first treatment applicator of the plurality of treatment applicators has a first size. A second treatment applicator of the plurality of treatment applicators has a second size. The first size is different from the second size. Each treatment applicator of the plurality of treatment applicators includes a substrate, a treatment layer coupled to the substrate, and a backing coupled to the treatment layer. The treatment layer includes a treatment agent.

In an example of the first aspect, each treatment applicator includes a front flap to extend over a front of a toenail.

In another example of the first aspect and the above examples, each treatment applicator includes a side flap to extend over skin at a side of a toenail.

In a further example of the first aspect and the above examples, each treatment applicator includes a rear flap to extend over a bed of a toenail.

In an additional example of the first aspect and the above examples, the method further includes a third treatment applicator of the plurality of treatment applicators having a third size different from the first and second sizes.

In another example of the first aspect and the above examples, the plurality of treatment applicators are disposed on a card, the card forming the backing.

In a further example of the first aspect and the above examples, the plurality of treatment applicators are disposed in a packet.

In an additional example of the first aspect and the above examples, the treatment agent includes an antifungal agent. For example, the antifungal agent includes lema oil, alkyl dimethyl benzyl ammonium chloride, onymyrrhe, myrrh extract, byungpul extract, manuka oil, tea tree oil, zinc undecylenate and undecylenic acid, mentholated ointments such as menthol, eucalyptus oil, and camphor, snakeroot extract, lavender oil, petitgrain oil, clary sage oil, ylang oil, jasmine oil, oregano oil, garlic extracts, or any combination thereof.

In another example of the first aspect and the above examples, the treatment agent include a health promoting agent. For example, the health promoting agent includes vitamin E, jojoba oil, coconut oil, olive oil, sweet almond oil, rice bran oil, shea butter, avocado oil, bee wax, lemon oil, argan oil, lanolin, aloe vera, or any combination thereof.

In a further example of the first aspect and the above examples, the treatment agent includes a transfer agent. For example, the transfer agent includes propylene glycol, glycerin, mineral oil, a surfactant such as sodium dodecylbenzene sulfonate, or any combination thereof.

In an additional example of the first aspect and the above examples, the method further includes an adhesive layer disposed on a surface of the substrate, the treatment layer secured to the adhesive layer. For example, the adhesive layer includes an acrylic, butyl rubber, ethylene-vinyl acetate, natural rubber, nitriles, silicone rubbers, styrene block polymers, styrene-ethylene/butylene-styrene (SEBS), styrene-ethylene/propylene (SEP), styrene-isoprene-styrene (SIS), vinyl ethers, or any combination thereof. In another example, the adhesive layer includes a tackifier. For example, the tackifier includes rosins derivates, terpenes and modified terpenes, aliphatic, cycloaliphatic or aromatic resins (C5 aliphatic resins, C9 aromatic resins, or C5/C9 aliphatic/aromatic resins), hydrogenated hydrocarbon resins, terpene-phenol resins (TPR) or any combination thereof.

In another example of the first aspect and the above examples, the treatment layer includes a tacky material to adhere to a nail. For example, the tacky material includes an acrylic, butyl rubber, ethylene-vinyl acetate, natural rubber, nitriles, silicone rubbers, styrene block polymers, styrene-ethylene/butylene-styrene (SEBS), styrene-ethylene/propylene (SEP), styrene-isoprene-styrene (SIS), vinyl ethers, or any combination thereof. In another example, the tacky material includes a tackifier. For example, the tackifier includes rosins derivates, terpenes and modified terpenes, aliphatic, cycloaliphatic or aromatic resins (C5 aliphatic resins, C9 aromatic resins, or C5/C9 aliphatic/aromatic resins), hydrogenated hydrocarbon resins, terpene-phenol resins (TPR) or any combination thereof.

In a second aspect, a method for treating a nail includes removing a set of treatment applicators from a packet, the set of treatment applicators having a shape indicative of a nail, a first treatment applicator of the set of treatment applicators having a first size, a second treatment applicator of the set of treatment applicators having a second size, the first size being different from the second size, each treatment applicator of the set of treatment applicators including a substrate, a treatment layer coupled to the substrate, and a backing coupled to the treatment layer, the treatment layer including a treatment agent; selecting a treatment applicator from the set, the treatment applicator having a select size; removing the backing from the treatment application; and applying the treatment applicator to a nail associated with the select size.

In an example of the second aspect, the treatment agent is an antifungal agent.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

What is claimed is:

1. A treatment kit comprising:
a plurality of treatment applicators having a shape indicative of a nail, a first treatment applicator of the plurality of treatment applicators having a first size, a second treatment applicator of the plurality of treatment applicators having a second size, the first size being different from the second size;
each treatment applicator of the plurality of treatment applicators including a substrate, a treatment layer coupled to the substrate, and a backing coupled to the treatment layer, the treatment layer including a treatment agent.

2. The treatment kit of claim 1, wherein the each treatment applicator includes a front flap to extend over a front of a toenail.

3. The treatment kit of claim 1, wherein the each treatment applicator includes a side flap to extend over skin at a side of a toenail.

4. The treatment kit of claim 1, wherein the each treatment applicator includes a rear flap to extend over a bed of a toenail.

5. The treatment kit of claim 1, further comprising a third treatment applicator of the plurality of treatment applicators having a third size different from the first and second sizes.

6. The treatment kit of claim 1, wherein the plurality of treatment applicators are disposed on a card, the card forming the backing.

7. The treatment kit of claim 1, wherein the plurality of treatment applicators are disposed in a packet.

8. The treatment kit of claim 1, wherein the treatment agent includes an antifungal agent.

9. The treatment kit of claim 8, wherein the antifungal agent includes lema oil, alkyl dimethyl benzyl ammonium chloride, onymyrrhe, myrrh extract, byungpul extract, manuka oil, tea tree oil, zinc undecylenate and undecylenic acid, mentholated ointments such as menthol, eucalyptus oil, and camphor, snakeroot extract, lavender oil, petitgrain oil, clary sage oil, ylang ylang oil, jasmine oil, oregano oil, garlic extracts, or any combination thereof.

10. The treatment kit of claim 1, wherein the treatment agent include a health promoting agent.

11. The treatment kit of claim 10, wherein the health promoting agent includes vitamin E, jojoba oil, coconut oil, olive oil, sweet almond oil, rice bran oil, shea butter, avocado oil, bee wax, lemon oil, argan oil, lanolin, aloe vera, or any combination thereof.

12. The treatment kit of claim 1, wherein the treatment agent includes a transfer agent.

13. The treatment kit of claim 12, wherein the transfer agent includes propylene glycol, glycerin, mineral oil, a surfactant such as sodium dodecylbenzene sulfonate, or any combination thereof.

14. The treatment kit of claim 1, further comprising an adhesive layer disposed on a surface of the substrate, the treatment layer secured to the adhesive layer.

15. The treatment kit of claim 14, wherein the adhesive layer includes an acrylic, butyl rubber, ethylene-vinyl acetate, natural rubber, nitriles, silicone rubbers, styrene block polymers, styrene-ethylene/butylene-styrene (SEBS), styrene-ethylene/propylene (SEP), styrene-isoprene-styrene (SIS), vinyl ethers, or any combination thereof.

16. The treatment kit of claim 14, wherein the adhesive layer includes a tackifier.

17. The treatment kit of claim 16, wherein the tackifier includes rosins derivates, terpenes and modified terpenes, aliphatic, cycloaliphatic or aromatic resins (C5 aliphatic resins, C9 aromatic resins, or C5/C9 aliphatic/aromatic resins), hydrogenated hydrocarbon resins, terpene-phenol resins (TPR) or any combination thereof.

18. The treatment kit of claim 1, wherein the treatment layer includes a tacky material to adhere to a nail.

19. The treatment kit of claim 18, wherein the tacky material includes an acrylic, butyl rubber, ethylene-vinyl acetate, natural rubber, nitriles, silicone rubbers, styrene block polymers, styrene-ethylene/butylene-styrene (SEBS), styrene-ethylene/propylene (SEP), styrene-isoprene-styrene (SIS), vinyl ethers, or any combination thereof.

20. The treatment kit of claim 18, wherein the tacky material includes a tackifier.
